(19) United States
(12) Patent Application Publication
(10) Pub. No.: US 2016/0297131 A1
(43) Pub. Date: Oct. 13, 2016

(54) Hydrogel Microparticles via Soft Robotics Micromold (SRM) for In Vitro Cell Culture

(71) Applicant: THE TEXAS A&M UNIVERSITY SYSTEM, College Station, TX (US)

(72) Inventors: Jun Kameoka, College Station, TX (US); Po-Jung Huang, College Station, TX (US); Roland Kaunas, College Station, TX (US); Carl A. Gregory, Belton, TX (US)

(21) Appl. No.: 15/093,356

(22) Filed: Apr. 7, 2016

Related U.S. Application Data
(60) Provisional application No. 62/144,037, filed on Apr. 7, 2015.

(51) Int. Cl. B29C 47/00; A61L 27/38; A61L 27/54; B29C 33/40; G01N 33/50; B29C 47/30; B29C 33/38; A61L 27/52; A61L 27/26

(52) U.S. Cl. CPC B29C 47/003; A61L 27/52; A61L 27/38; A61L 27/54; A61L 27/26; G01N 33/5082; B29C 47/0004; B29C 47/30; B29C 33/3842; B29C 33/405; A61L 2300/64; B29K 2105/0061

(57) ABSTRACT

The present invention includes a mold and a method for producing an engineered tissue construct comprising: providing a mold comprising one or more openings, the mold being at least partially elastic and the one or more openings having a pre-determined shape; and extruding through the one or more openings in the mold a biocompatible gel-forming macromer to form a hydrogel using a mechanical force sufficient to extrude the biocompatible gel-forming macromer.

### CROSS-REFERENCE TO RELATED APPLICATIONS

This application claims benefit of U.S. Provisional Application Ser. No. 62/144,037 filed Apr. 7, 2015 which is incorporated herein by reference in its entirety.

### TECHNICAL FIELD OF THE INVENTION

The present invention relates in general to the field of biocompatible hydrogels, and more particularly, to hydrogel microparticles made using a soft robotics micromold (SRM) for in vitro cell culture.

### STATEMENT OF FEDERALLY FUNDED RESEARCH

Not Applicable.

### BACKGROUND OF THE INVENTION

Without limiting the scope of the invention, its background is described in connection with hydrogels.

United States Patent Application Publication No. 2011/0171712 filed by Rivron, et al., is entitled “Self-Assembling Tissue Modules.” Briefly, these applicants described a method for constructing cellular aggregates in vitro and their use in methods for producing 3D-tissue constructs in a modular way. The method for in vitro production of a tissue construct comprises: a) combining living cells to form supracellular aggregates using spatial confinement; b) combining two or more of the supracellular aggregates in a mold or on a biomaterial; c) applying conditions that induce self-assembly within the combined supracellular aggregates to obtain the tissue construct; and d) applying conditions that induce tissue morphogenesis in the tissue construct.

United States Patent Application Publication No. 2008/0206308, filed by Jabbari, et al., entitled “Hydrogel Porogents for Fabricating Biodegradable Scaffolds.” Briefly, these applicants teach hydrogel microparticles with entrapped liquid used as a porogen to reproducibly form interconnected pore networks in a porous scaffold. In one embodiment, a biodegradable unsaturated polymer, a crosslinking agent, and a porogen comprising biodegradable hydrogel microparticles are mixed together and allowed to form a porous scaffold in a mold or in a body cavity. Example biodegradable unsaturated polymers include poly(propylene fumarate) and poly(e-caprolactone-fumarate). The crosslinking agent may be a free radical initiator, or may include a free radical initiator and a monomer capable of addition polymerization. Example hydrogel microparticles include uncrosslinked or crosslinked collagen, an uncrosslinked or crosslinked collagen derivative, and an uncrosslinked or crosslinked synthetic biodegradable polymer such as oligo(poly(ethylene glycol) fumarate).

### SUMMARY OF THE INVENTION

In one embodiment, the present invention includes a method for producing an engineered tissue construct comprising: providing a mold comprising one or more openings, the mold being at least partially elastic and the one or more openings having a pre-determined shape; and extruding through the one or more openings in the mold, a biocompatible gel-forming macromer to form a hydrogel using a mechanical force sufficient to extrude the hydrogel. In one aspect, the hydrogel is a collagen microparticle that can be used for arthritis treatment. In one aspect, the hydrogel is very economical and can be easily commercialized for many different applications, such as tissue engineering, dermal fillers or cartilage damage. In one aspect, the mechanical force is defined further as at least one of a vertical or a horizontal mechanical force. In one aspect, the method further comprises the step of isolating the extruded biocompatible hydrogel forms from the mold, wherein deformation of the mold causes the hydrogel in the pre-determined shape to be released from the mold. In another aspect, the mold comprises at least one of paper, cellulose, polydimethylsiloxane, rubber, plastic, polyethylene glycol, polytetrafluoroethylene (PTFE), polyphenyl ether polymers, modified polyphenyl ether polymers, poly(phenyl ether), or polyphenyl polyether. In another aspect, a mechanical force is exerted on a reservoir that comprises the biocompatible gel-forming macromer, wherein the mechanical force is at least one of direct mechanical, pneumatic, or hydraulic (water or oil) force. In another aspect, the pre-determined shape includes at least one of shapes that can be interlocked into larger forms, have male and female interlocking portions, interlocking bricks, tongue and groove, dovetail joints, irregular, triangular, square, rectangular, pyramidal, rhomboidal, cross-shaped, bullet-shaped, cubic shaped, a tetrapod, a multipod, an arbitrary and partially curved. In another aspect, the pre-determined shape is not spherical. In another aspect, the biocompatible gel-forming macromer is dextran, heparin, heparin sulfate, chondroitin sulfate, hyaluronic acid, alginate, gelatin, collagen, albumin, ovalbumin, polyaminoacid, a single, oligomeric or polymeric residue of glycolic acid, lactic acid, caprolactone, butyrolactone, valerolactone, or carbonate. In one aspect, the biocompatible gel-forming macromer is aligned into fibrils after formation into the hydrogel. In one aspect, the hydrogel further comprises one or more cells. In one aspect, the hydrogel further comprises one or more cells or tissues selected from at least one of pancreatic beta cells, pancreatic islets, chondrocytes, bone marrow, hepatocytes, pluripotent stem cells, totipotent stem cells, hematopoietic cells, mesenchymal stem cells, neural stem cells, cardiac stem cells, kerotinocytes, fibroblasts, ligament cells, endothelial cells, lung cells, epithelial cells, smooth muscle cells, cardiac muscle cells, skeletal muscle cells, nerve cells, kidney cells, bladder cells, urothelial cells, skin cells, neurons, Schwann cells, thyroid cells, reproductive cells, cancer cells or bone-forming cells. In another aspect, the hydrogel further comprises one or more cells that are autologous to a subject for implantation, drug screening or tumor drug screening. In another aspect, the hydrogel further comprises one or more biologically active materials selected from at least one of a synthetic inorganic compound, an organic compound, a protein, a peptide, a polysaccharide, a lipid, a ganglioside, a nucleic acid, a growth factor, an antibody, a receptor, a lectin, a biological scaffold, a drug, a chemical, or a chemotactic factor. In another aspect, the hydrogel further comprises one or more biologically active materials selected from at least one of a synthetic inorganic compound, an organic compound, a protein, a peptide, a polysaccharide, a lipid, a ganglioside, a nucleic acid, a growth factor, an antibody, a receptor, a lectin, a biological scaffold, a drug, a chemical, or a chemotactic factor covalently bound to the hydrogel. In another aspect, the method further comprises the step of imaging a shape for insertion of the hydrogel, making a mold having the shape and size of the pre-determined shape, and extruding a hydrogel through the mold to form a hydrogel with the same shape and size of the pre-determined shape. In another aspect, the hydrogel is defined further as a first extruded hydrogel and is incubated with a first cell type and a second extruded hydrogel is incubated with a second cell type, and the first and second hydrogels are incubated together after isolation. In another aspect, the hydrogel is an extruded hydrogel, is isolated and polymer fibrils, electrospun fibrils, or glycosaminoglycans are added into the interstitial spaces between two or more extruded hydrogels. In another aspect, the hydrogel is an extruded hydrogel, is isolated and polymer fibrils, electrospun fibrils, or glycosaminoglycans are added into the interstitial spaces between two or more extruded hydrogels and fluid is pumped between the two or more extruded hydrogels to mimic interstitial flow. In another aspect, the hydrogel is an extruded hydrogel is further subjected to compressive or tensile forces. In another aspect, the hydrogel is defined further as two or more extruded hydrogels that are incubated in a rotating bioreactor. In another aspect, the hydrogel is defined further as two or more extruded hydrogels that are formed into a tissue system, wherein the tissue system mimics in vivo biological system, specific cell behaviors, cell migration, viability, angiogenesis, apoptosis, proliferation, differentiation, gene expression, protein synthesis, protein secretion, tissue formation, cancer drug screening and cancer drug screening.

In another embodiment, the present invention includes a method for making an hydrogel having a pre-determined shape comprising: providing a mold comprising one or more openings, the mold being at least partially elastic and the one or more openings having the pre-determined shape; extruding through the one or more openings in the mold a biocompatible gel-forming macromer to form a hydrogel using a mechanical force sufficient to extrude the biocompatible gel-forming macromer; and isolating the extruded biocompatible hydrogel forms from the mold, wherein deformation of the mold causes the hydrogel in the predefined shape to be released from the mold. In one aspect, the mechanical force is defined further as at least one of a vertical or a horizontal mechanical force. In one aspect, the method further comprises the step of isolating the extruded biocompatible hydrogel forms from the mold, wherein deformation of the mold causes the hydrogel in the pre-determined shape to be released from the mold. In another aspect, the mold comprises at least one of paper, cellulose, polydimethylsiloxane, rubber, plastic, polyethylene glycol, polytetrafluoroethylene (PTFE), polyphenyl ether polymers, modified polyphenyl ether polymers, poly(phenyl ether), or polyphenyl polyether. In another aspect, a mechanical force is exerted on a reservoir that comprises the biocompatible gel-forming macromer, wherein the mechanical force is at least one of direct mechanical, pneumatic, or hydraulic (water or oil) force. In another aspect, the pre-determined shape includes at least one of shapes that can be interlocked into larger forms, have male and female interlocking portions, interlocking bricks, tongue and groove, dovetail joints, irregular, triangular, square, rectangular, pyramidal, rhomboidal, cross-shaped, bullet-shaped, cubic shaped, a tetrapod, a multipod, an arbitrary and partially curved. In another aspect, the pre-determined shape is not spherical. In another aspect, the biocompatible gel-forming macromer is dextran, heparin, heparin sulfate, chondroitin sulfate, hyaluronic acid, alginate, gelatin, collagen, albumin, ovalbumin, polyaminoacid, a single, oligomeric or polymeric residue of glycolic acid, lactic acid, caprolactone, butyrolactone, valerolactone, or carbonate. In one aspect, the biocompatible gel-forming macromer is aligned into fibrils after formation into the hydrogel. In one aspect, the hydrogel further comprises one or more cells. In one aspect, the hydrogel further comprises one or more cells or tissues selected from at least one of pancreatic beta cells, pancreatic islets, chondrocytes, bone marrow, hepatocytes, pluripotent stem cells, totipotent stem cells, hematopoietic cells, mesenchymal stem cells, neural stem cells, cardiac stem cells, kerotinocytes, fibroblasts, ligament cells, endothelial cells, lung cells, epithelial cells, smooth muscle cells, cardiac muscle cells, skeletal muscle cells, nerve cells, kidney cells, bladder cells, urothelial cells, skin cells, neurons, Schwann cells, thyroid cells, reproductive cells, or bone-forming cells. In another aspect, the hydrogel further comprises one or more cells that are autologous to a subject for implantation, drug screening or tumor drug screening. In another aspect, the hydrogel further comprises one or more biologically active materials selected from at least one of a synthetic inorganic compound, an organic compound, a protein, a peptide, a polysaccharide, a lipid, a ganglioside, a nucleic acid, a growth factor, an antibody, a receptor, a lectin, a biological scaffold, a drug, a chemical, or a chemotactic factor. In another aspect, the hydrogel further comprises one or more biologically active materials selected from at least one of a synthetic inorganic compound, an organic compound, a protein, a peptide, a polysaccharide, a lipid, a ganglioside, a nucleic acid, a growth factor, an antibody, a receptor, a lectin, a biological scaffold, a drug, a chemical, or a chemotactic factor covalently bound to the hydrogel. In another aspect, the method further comprises the step of imaging a shape for insertion of the hydrogel, making a mold having the shape and size of the pre-determined shape, and extruding a hydrogel through the mold to form a hydrogel with the same shape and size of the pre-determined shape. In another aspect, the hydrogel is defined further as a first extruded hydrogel is incubated with a first cell type and a second extruded hydrogel is incubated with a second cell type, and the first and second hydrogels are incubated together after isolation. In another aspect, the hydrogel is an extruded hydrogel that is isolated and polymer fibrils, electrospun fibrils, or glycosaminoglycans are added into the interstitial spaces between two or more extruded hydrogels. In another aspect, the hydrogel is an extruded hydrogel that is isolated and polymer fibrils, electrospun fibrils, or glycosaminoglycans are added into the interstitial spaces between two or more extruded hydrogels and fluid is pumped between the two or more extruded hydrogels to mimic interstitial flow. In another aspect, the hydrogel is an extruded hydrogel that is further subjected to compressive or tensile forces. In another aspect, the hydrogel is defined further as two or more extruded hydrogels that are incubated in a rotating bioreactor. In another aspect, the hydrogel is defined further as two or more extruded hydrogels that are formed into a tissue system, wherein the tissue system mimics in vivo biological system, specific cell behaviors, cell migration, viability, angiogenesis, apoptosis, proliferation, differentiation, gene expression, protein synthesis, protein secretion, tissue formation, and cancer drug screening.

Yet another embodiment of the present invention includes a soft robotics micromold comprising: a particle layer comprising one or more openings having a predetermined shape for making a hydrogel, wherein the particle layer maintains the predetermined shape but is also flexible; a fluid-channel layer disposed adjacent the particle layer, wherein the fluid-channel layer is constructed by a three-dimensional printer and comprises an elastic materials, the fluid channel layer comprising one or more channels that can be filled with a gas or a liquid that can displace a polymeric shape formed in the one or more openings; and a bottom layer disposed adjacent the fluid-channel layer and opposite the particle layer, wherein the bottom layer is less elastic than the particle layer. In one aspect, the particle layer comprises a photopolymerizable polymer. In another aspect, the fluid-channel layer comprises at least one or a paper, cellulose, polydimethylsiloxane, rubber, plastic, polyethylene glycol, polytetrafluoroethylene (PTFE), polyphenyl ether polymers, modified polyphenyl ether polymers, poly(phenyl ether), or polyphenyl polyether. In another aspect, a hydrogel formed in the one or more openings comprises at least one of dextran, heparin, heparin sulfate, chondroitin sulfate, hyaluronic acid, alginate, gelatin, collagen, albumin, ovalbumin, polyaminoacid, a single, oligomeric or polymeric residue of glycolic acid, lactic acid, caprolactone, butyrolactone, valerolactone, or carbonate. In one aspect, the hydrogel further comprises one or more cells or tissues selected from at least one of pancreatic beta cells, pancreatic islets, chondrocytes, bone marrow, hepatocytes, pluripotent stem cells, totipotent stem cells, hematopoietic cells, mesenchymal stem cells, neural stem cells, cardiac stem cells, kerotinocytes, fibroblasts, ligament cells, endothelial cells, lung cells, epithelial cells, smooth muscle cells, cardiac muscle cells, skeletal muscle cells, nerve cells, kidney cells, bladder cells, urothelial cells, skin cells, neurons, Schwann cells, thyroid cells, reproductive cells, cancer cells or bone-forming cells.

Another embodiment of the present invention includes a method of making a soft robotics micromold comprising: providing a particle layer comprising one or more openings having a predetermined shape for making a hydrogel, wherein the particle layer maintains the predetermined shape but is also flexible; depositing a fluid-channel layer disposed adjacent the particle layer, wherein the fluid-channel layer is constructed by a three-dimensional printer and comprises an elastic materials, the fluid channel layer comprising one or more channels that can be filled with a gas or a liquid that can displace a polymeric shape formed in the one or more openings; and attaching a bottom layer to the fluid-channel layer and opposite the particle layer, wherein the bottom layer is less elastic than the particle layer. In one aspect, the particle layer comprises a photopolymerizable polymer. In another aspect, the fluid-channel layer comprises at least one or a paper, cellulose, polydimethylsiloxane, rubber, plastic, polyethylene glycol, polytetrafluoroethylene (PTFE), polyphenyl ether polymers, modified polyphenyl ether polymers, poly(phenyl ether), or polyphenyl polyether. In another aspect, a hydrogel formed in the one or more openings comprises at least one of dextran, heparin, heparin sulfate, chondroitin sulfate, hyaluronic acid, alginate, gelatin, collagen, albumin, ovalbumin, polyaminoacid, a single, oligomeric or polymeric residue of glycolic acid, lactic acid, caprolactone, butyrolactone, valerolactone, or carbonate. In one aspect, the hydrogel further comprises one or more cells or tissues selected from at least one of pancreatic beta cells, pancreatic islets, chondrocytes, bone marrow, hepatocytes, pluripotent stem cells, totipotent stem cells, hematopoietic cells, mesenchymal stem cells, neural stem cells, cardiac stem cells, kerotinocytes, fibroblasts, ligament cells, endothelial cells, lung cells, epithelial cells, smooth muscle cells, cardiac muscle cells, skeletal muscle cells, nerve cells, kidney cells, bladder cells, urothelial cells, skin cells, neurons, Schwann cells, thyroid cells, reproductive cells, cancer cells or bone-forming cells.

### BRIEF DESCRIPTION OF THE DRAWINGS

For a more complete understanding of the features and advantages of the present invention, reference is now made to the detailed description of the invention along with the accompanying figures and in which:

FIGS. 1a to 1d show a step-by-step summary of the Soft Mechanic Device (SMD) of the present invention. FIG. 1a shows the soft mechanic device is composed by syringe, mold and pneumonic system (FIG. 1b), (FIG. 1d) mold and pneumonic system. The main material of mold is PDMS thin film, which was printed with different shapes of pattern. The second layer—Channel, is made by ECOFLEX®. The composite-ECOFLEX® and paper is the main material for the third layer. FIG. 1c shows the hydrogel after injection, the whole system bends and the pattern deformation pushes out the hydrogel with different shapes.

FIGS. 2a to 2d summarize the deformation of pattern on the mold after injecting, in this case, with air. The variation of (FIG. 2a) depth (FIG. 2b) width during injecting different volume of air. Collagen particles are released by (FIG. 2c) soft mechanic system (FIG. 2d) hand pushing.

FIG. 3 shows the basic outline of the use of the Soft Robotics Micromold (SRM).

FIGS. 4a to 4i show the morphology of various Collagen particles with different shapes are made by soft pneumonic system. (FIG. 4a) square shape; (FIG. 4b) cross and square shape; (FIG. 4c) cross shape; (FIG. 4d) top view of mold for bullet shape; (FIG. 4e) side view of mold for bullet shape; (FIG. 4f) bullet shape; (FIG. 4g) mold for roller slide shape; (FIG. 4h) the micromold and PEGDA microparticles with roller-slide shape are shown; and (FIG. 4i) Collagen particles pass through the syringe (including needle), and the shape of collagen particle are not interrupted by passing though syringe.

FIGS. 5a and 5b show that a collagen morphology with a high aspect ratio (FIG. 5a) top view, (FIG. 5b) side view.

FIGS. 6Aa to 6Aa-4 and 6Bb-1 to 6Bb-4 show a second harmonic generation image and alignment index for the various constructs. FIG. 6Aa shows collagen microparticles with cross shape as follows: FIG. 6Aa-1 Collagen Fibril Alignment shown in three directions, Alignment index (AI)=1.5713; FIG. 6Aa-2 Collagen Fibril Alignment at corner I, AI=1.3514; FIG. 6Aa-3 Collagen Fibril Alignment at corner II, AI=1.5491; FIG. 6Aa-4 Collagen Fibril Alignment at center, AI=1.1354; FIG. 6Bb-1 Collagen Fibril Alignment in donut shape at center, AI=1.1747; FIG. 6Bb-2 Collagen Fibril Alignment in Donut Shape at Edge I, AI=1.6957; FIG. 6Bb-3 Collagen Fibril Alignment in donut shape at edge II, AI=1.5199; FIG. 6Bb-4 Collagen Fibril Alignment in donut shape at edge III, AI=1.5422.

FIGS. 7a to 7e show cell-based encapsulated collagen microparticles. FIG. 7a shows Cell Viability, FIG. 7b shows optical image for cell encapsulated collagen microparticles. FIG. 7c shows fluorescent image of collagen microparticles for 7-day incubation. FIG. 7d shows a second harmonic generation image for MDA-MS-231 cells and collagen, Red: collagen fiber, Green: MDA-MS-231. FIG. 7e shows the fiber alignment of collagen after 7-day incubation.

FIG. 8 shows the progress of Cell-Based Encapsulated Collagen Microparticles over various days.

FIGS. 9a to 9f show one implementation of the present invention. FIG. 9a is a schematic diagram of soft micromold device, which compose micro-scale surface template for molding (PDMS thin film), pneumatic actuation to deform micromold and extract collagen microparticles (Ecoflex layer), and stress layer (cloth embedded Ecoflex composite), and the position 1 to 5 is named from near air inlet to far-air inlet, respectively. FIG. 9b shows the deformation of micromold on SRM platform with pneumatic actuation from 1.0 atm to 2.0 atm. (Scale bar is 100 μm for all images). FIG. 9c is a schematic diagram of 3D deformation of micromold; FIG. 9d shows the deformation of soft micromold at (d) x-width; FIG. 9e shows the y-width; and FIG. 9f the z-direction based on the micromold location on SMM devices shown in FIG. 9a.

FIGS. 10a to 10o, show the characterization of collagen microstructures made by SMM device. Microscopic images of microstructures and molds for (FIGS. 10a-10b) cross column; (FIGS. 10c-10d) pentagonal column; (FIGS. 10e-10f) hollow circular cylinder; (FIGS. 10g-10h) triangular column; (FIGS. 10i-10j), square pad, respectively. The thickness of these microstructures are set to 100 μm. (FIG. 10k) Duplication accuracy as function of dimensions of micro molds. (FIG. 10l) Second harmonic generation microscopic image of collagen fibers in micro cross column. Scale bar is 100 μm for FIGS. 10a, 10c, 10d, 10g, 10i. (FIG. 10m) Dimensions of collagen micro-cubes as function of times after productions. (FIG. 10o) Collagen microstructures extracted from mold patterns by SRM and manual pushing. The graph for microstructure extraction success rate between SRM device and manual pushing approaches. The scale bar is 500 μm.

FIG. 11 is a graph that compares the glucose concentration and the insulin secretion using suspended beta cells, encapsulated beta cells in 1 mm collagen particles and encapsulated beta cells in 0.5 mm collagen particles.

FIG. 12 is a schematic representation of one example of a SRM producing 30,000 microparticles.

FIGS. 13A-a to 13A-d show a schematic diagram of fabrication process for Soft micro mold device. Fabricating process of (FIG. 13A-a) surface template layer (FIG. 13A-b) pneumatic system layer (FIG. 13A-c) barrier layer. (FIG. 13A-d) The bonding process of these three layers. (FIG. 13A-e) Photographic image of SMM device with TYGON® tubing.

FIGS. 13B-a to 13B-c, show the Particles Releasing Process (FIG. 13B-a) Collagen microstructure fabrication process. Filling collagen solution into surface templates and curing at 37° C. (FIG. 13B-b) Collagen microstructure extraction process with pneumatic actuation. This is a side view as shown by the arrow in FIGS. 13B-a to 13B-c show a microscopic image of micro scale surface templates of cross prism shape. (FIG. 13B-c) shows the mold image with collagen.

FIGS. 14A and 14B show an FE simulation of deformation of SRM: FIG. 14A Extension in circumferential direction due to inner pressures; FIG. 14B Deformation in portion of SRM due to air pressure at 2 atm (red=max principal stretch, blue=zero stretch.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Hydrogels are particularly attractive for many biomedical applications such as tissue engineering because their biodegradable nature and high porosity allows adequate transport of nutrients and oxygen to cells. The present inventors demonstrate herein a novel method for producing hydrogel microparticles via a soft robotics micromold (SRM). One important feature is that the shape of microparticles is not just spherical. SRM integrated pneumatic channel and Particle Replication In Non-wetting Templates (PRINT®) were used in order to make a hydrogel with 2D and 3D structure, having user-controlled or pre-determined shapes. Ecoflex® is one material for use with the SRM device and Ecoflex is easy to deform the structure when the force is generated inside a channel. When air was injected into the channel, the micromold starts deforming, which causes a difference in the mechanical properties between a top and a bottom layer of the mold. For example, the width of pattern can increase from 500 μm to 550 μm and the depth of pattern decreases from 80 μm to 70 μm. In one example, phosphate buffered saline (PBS) can diffuse inside the mold and the hydrogel will release from the mold and even start floating. Finally, hydrogel particles will be pushed out from micromold either from the interior of the mold as a result of increased internal pressure, due to the decreasing size of micromold when released from a bent configuration, or both. Compared to conventional methods, SRM can be used to make different shapes of particles such as square, cross shape (2D structure), bullet, roller-slide shape (3D structure) successfully. The size and the shape can be pre-determined (or even printed) to be the pattern as the molds and/or the target application of the hydrogel (e.g., the shape of a wound).

In one non-limiting example, collagen is used as the basic substrate from which to make the hydrogel. For collagen type I microparticles made using the present invention, a fiber alignment was found in the microparticles, which was made by SRM. A Second Harmonic Generation (SHG) image shows alignment of the collagen fibers area in the specific edge, especially at the edge region. The inventors used Matlab, Fast Fourier Transfer (FFT), to quantize the level of alignment. Taking the collagen particles with a cross shape, as an example, an alignment index (AI) at the center part was 1.1354, which is random angle distribution. Moreover, the alignment index at the edge part was from 1.35 to 1.51, which indicates an aligned collagen was formed in the collagen microparticles of the present invention. When collagen particles were released from micromold, by force placed on the micromold, without affecting the collagen alignment. Surprisingly, an aligned collagen can be synthesized by SRM.

To apply in tissue engineering, cell-based collagen microparticles with different shapes were made in SRM. The inventors also introduced cells, e.g., MDA-MB-231 cells, into the microparticles. It was found that cells could survive in excess of 14 days when they were encapsulated in the collagen microparticles of the present invention. When cell-based collagen microparticles exist alone, the size of whole microparticles will start shrinking. On the other hand, when two cell-based collagen microparticles approached, intertumoral actions were triggered. These results indicated that SRM can be applied in tissue engineering because collagen microparticles with any specific 3D shapes and sizes can be synthesized, even when cells were encapsulated inside the collagen microparticles.

External cellular matrix (ECM) can typically provide structural and biochemical support to cells, which can be added to the hydrogel (or matrix) made herein. Therefore, the interaction between cells or tissues can be studied by building up the external cellular matrix (ECM), for example, when used to study cellular metastasis. Three-dimension cell culture system is an essential issue for tissue engineering because 3D environment can mimic and approach the environment of in vivo tissues. Normally, encapsulating cells in biopolymer is the most common method to investigate the intercellular interaction.

For use with the present invention, various examples of external cellular matrices (or hydrogels) can be made by existing methods including: (1) water-in-oil emulsion. (2) Continuous-flow lithography (CFL), (3) stop-flow lithography (SFL), (4) replica molding, and (5) Particle Replication In Non-wetting Templates (PRINT®). Droplet generation, gelation and extraction were integrated into a single chip of water-and-oil system^{[2]}. Therefore, hydrogel microspheres or cell-encapsulated microspheres can be generated by microfluidic chip rapidly. Importantly, the size of microsphere is uniform and controllable. The size can be controlled by adjusting the flow rate of aqueous phase (polymer solution). Generally, water-and-oil emulsion are typically used just to form sphere-like microparticles because the thermodynamics of sphere in the water-and-oil system is most stable than other shapes. The problem with this method is the great variability of the spherical particles, and the limitation on the shape of the particles (basically spherical, tear-shaped or spheroidal). Another important limitation of existing methods is that the oil-phase is hard to remove completely after collecting hydrogel microspheres. Yet another further limitation of existing technology is the toxicity of microspheres, which damage and affect the growth of cells for cell-encapsulated microsphere.

Continuous or stop flow lithography is the method for fabricating microparticles with different 2D shapes instead of spheres only. Polymer solution can be introduced into transparent microfluidic channel. Then, the mask is placed underneath of channel in order to make microparticles with different shapes, whose patterns are determined by mask. Moreover, the thickness of microparticles can be controlled by exposure time or the light with different wavelengths. Also, the size of microparticles making from continuous or stop flow lithography system is uniform. However, the material in continuous or stop lithography is limited because the whole process of fabrication is lithographic, requiring multiple steps and masks. Finally, the material in these two systems must be cross-linked by photopolymerization.

Replica mold and Particle Replication In Non-wetting Templates (PRINT®) break the limitation of material. Soft lithography is used in producing replica mold and PRINT® templates. The particle size can be monitored by mask design. No matter what kinds of method for cross-linking will be used to synthesize polymer, all material can be used in replica mold and PRINT® technology. However, microparticles will not keep the same shape as the mold completely, because microparticles need to release from replica mold or PRINT® templates via squeezing. A problem with this method is that the shape of microparticles is be damaged by the squeezing process.

Non-limiting examples of cells for use with the present invention include one or more different types of cells or precursors selected from, e.g., pancreatic beta cells, pancreatic islets, chondrocytes, bone marrow, hepatocytes, pluripotent stem cells, totipotent stem cells, hematopoietic cells, mesenchymal stem cells, neural stem cells, cardiac stem cells, kerotinocytes, fibroblasts, ligament cells, endothelial cells, lung cells, epithelial cells, smooth muscle cells, cardiac muscle cells, skeletal muscle cells, nerve cells, kidney cells, bladder cells, urothelial cells, skin cells, neurons, Schwann cells, thyroid cells, reproductive cells, or bone-forming cells. The skilled artisan will recognize that other types of individual types of cells, or combinations of different types of cells (e.g., a pancreatic islet) can also be used with the present invention.

### Example 1

### Soft Robotics Micromold (SRM)

The present inventors developed a new system—soft robotics micromold (SRM), which overcomes the problems for previous methods. The method presented here demonstrates: (1) highly reproducible generation of distinguishable microparticles with dimension 100 μm to 1,000 μm; (2) Controllable fabrication of sizes and shapes of microparticles; (3) patterning 3D pattern in SRM system; (4) No limitation of biopolymer; and (5) cells with 10⁸ to 10⁹ cells/cm³ can be encapsulated in biopolymer in SRM system. A MDA-231 breast cancer cell line was encapsulated in collagen microparticles and the intertumor interaction can be discovered in collagen particles. The inventors observed the situation in which collagen fibers were formed that breast cancer cells start migrating. The present invention provides for the first time a modular approach in tissue engineering via a simple tool for biological investigation of the behavior of cells in 3D environment.

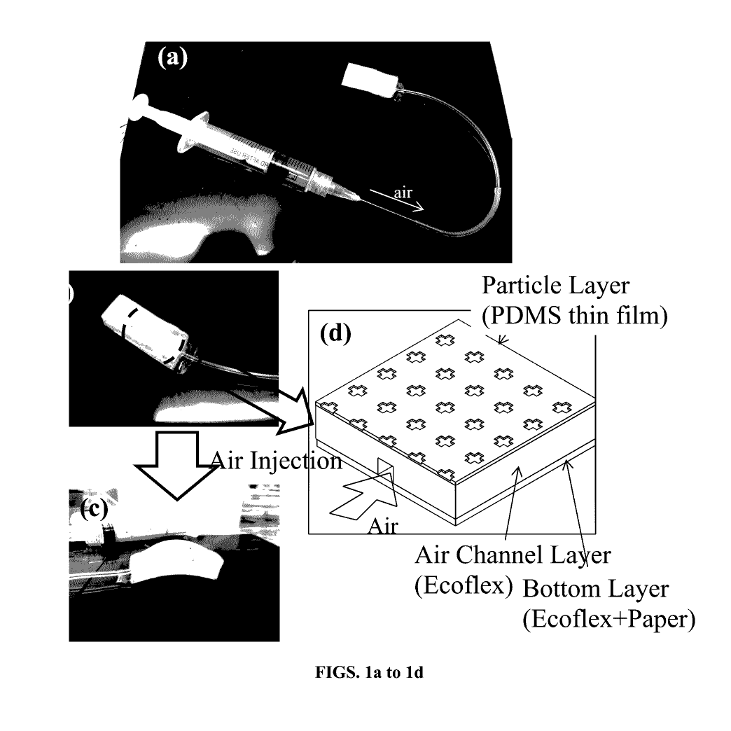

Soft Robotics Micromold Fabrication. Soft Robotics Micromold (SRM) was composed by three different layers: particle layer, air-channel layer, and bottom layer, respectively (FIGS. 1a-d). The particle layer is made as a top layer. The purpose of the particle later is that the collagen solution or collagen-cell mixture can be filled into the well for gelation. The pattern on the particle layer was also fabricated by lithographic process, a SU-8 2075 layer having a 100 μm thickness was spin-coated on top of silicon wafer. Then, PDMS was poured and spin-coated on the particle master mold in order to obtain PDMS thin film with different particle shapes. The thin film was cured at 65° C. for 2 hours.

The middle or fluid-channel layer is an air-channel layer whose pattern is constructed by 3D-printer. In one non-limiting example, the channel is 1.5 mm-width and 3.0 mm-height. The main material is ECOFLEX-30®, which is super-soft silicones rubber. Uncured ECOFLEX® polymer solution was spread over the 3D-printer patterned plastic mold and cured at room temperature for 4 hours. While the bottom layer is for fixed layer, which is composed by paper-embedded ECOFLEX® thin film. The thickness of composite film is 200 μm making by spin coating. Afterwards, uncured ECOFLEX® was used to connect these three layers (particle layer, air-channel layer, and bottom layer) and then this whole device was cured at 65° C. for 2 hours. Any material similar to ECOFLEX® can be used so long as it has similar flexibility, e.g., plastics, rubber, or composite materials or polymers.

Preparation of Alginate, PEG-diacrylate (PEGDA), and Collagen Microparticles with Different Shapes. To create alginate with different shapes via SRM system, each concentration for each batch was 1% alginate sodium solution. Next, the filter paper, which was emerged into Calcium chloride (0.1%), was placed on SRM system and then the Calcium chloride solution can diffuse into the wells for cross-linking. The whole device was put into an incubator for 10 minutes at 37° C. To synthesize PEGDA microparticles, PEGDA was mixed with a photoinitiator and then filled into the SRM. Then, the whole device was placed under a UV lamp for cross-linking. One minute later, the micromold was placed in a PBS solution in order to liberate the PEGDA microparticles from the micromold. All alginate and PEGDA microparticles could be released from mold by pneumatic system.

Cell Culture. The human breast cancer cell line, MDA-MB-231 expressing Green Fluorescent Protein (GFP) was cultured in Dulbecco's Minimal Essential Medium (DMEM)/F12 medium supplemented with 10% fetal bovine serum (FBS). Cells were cultured in a humidified 5% CO₂/95% air incubator at 37° C. Cells were removed with trypsin-EDTA from the culture dish and centrifuged. The concentration of MDA-MB-231 cells was 10⁸ to 10⁹ cells/mL.

Preparation of Collagen/Cell-Encapsulated Particles with Different Shapes. To fabricate the microscale collagen hydrogel with different shapes, each concentration for each batch was 3.5 mg/mL. The pH value of collagen type I solution was adjusted to 7.4. Next, the collagen solution can be poured and spread over the whole area in order to fill into wells completely. As the final step, the whole device can be placed into incubator 1 hour for gelation. The shape in this paper was designed as square (300 μm×300 μm, and 500 μm×500 μm), cross (300 μm×300 μm, and 500 μm×500 μm), and bullet shapes (Diameter: 1 mm, Depth: 1 mm). After gelation, the whole device can be fixed and immersed into PBS. The deformation of soft mechanic device was generated by injecting air into air channel. The collagen microparticle can be released from mold. For cell-encapsulated collagen microparticles, the human breast cancer cell line MDA-MB-231 expressing Green Fluorescent Protein (GFP) (1×10⁸ cells/mL), which cultured in Dulbecco's Minimal Essential Medium (DMEM), was mixed with collagen type I solution at concentration of 3.5 mg/mL. Cell-encapsulated collagen microparticles was cultured in a humidified 5% CO₂/95% air incubator at 37° C. These microparticles can be launched in Dulbecco's Minimal Essential Medium and cultured in incubator at the same condition as fabrication of cell-encapsulated microparticles.

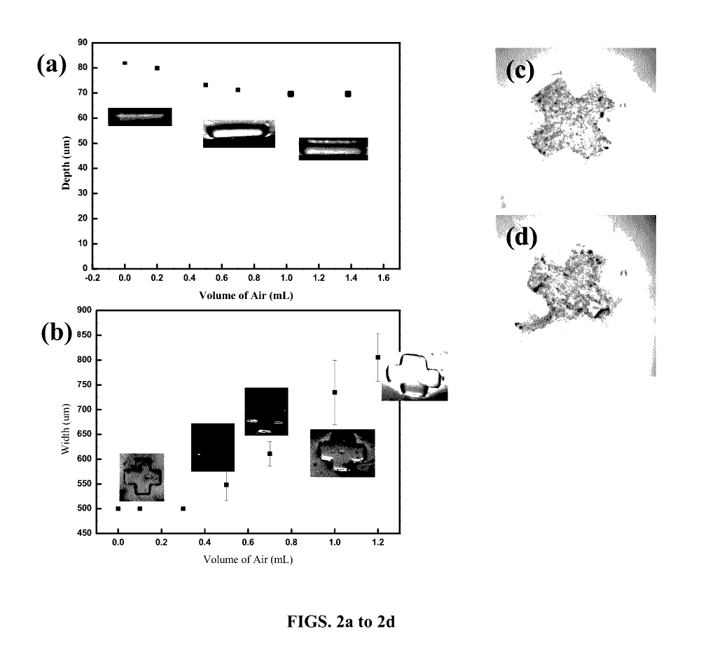

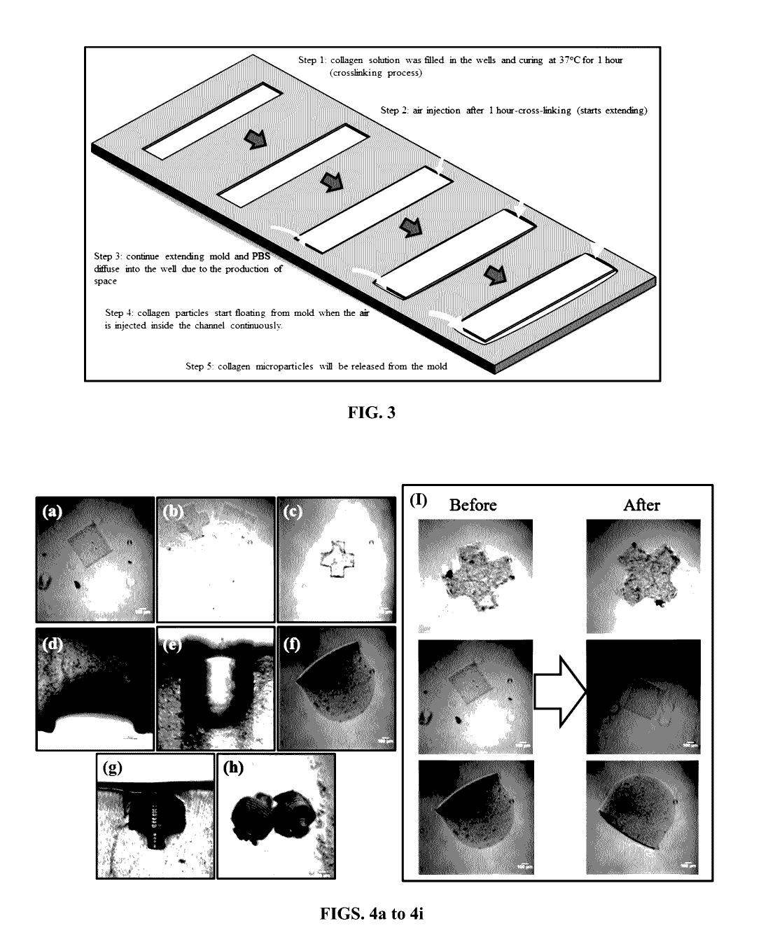

Collagen Microparticles Formation. A schematic diagram and photography of the soft mechanic device are shown in FIG. 1a to FIG. 1d, respectively. The pre-mixture of collagen or collagen-cell solution was expanded all of wells (FIG. 1d) on the PDMS thin film. Soft mechanic device needs to operate under ice bath in order to prevent the gelation of collagen solution. Once the collagen solution can be filled into the wells completely, the whole device can be placed into incubator and incubated at 37° C. in the humidified 5% CO₂ for 1 hour. After gelation, this device was immersed in PBS for 5 minutes. Next, the air in syringe would be injected into the channel in order to deform soft mechanic device. The deformation of soft mechanic device was shown in FIGS. 1b and 1c. When the air in syringe was not injected into the channel, the surface of soft mechanic device still maintained flat (FIG. 1b). After air injection, the deformation was formed by the differences between top layer (particle layer) and bottom layer (fixed layer). Therefore, the size of pattern will be changed due to deformation. FIGS. 2a to 2d show the results of pattern deformation. When the volume of air increases from 0.0 to 0.3 mL, the width of pattern is still the same size. As the volume was amplified to 0.5 mL, the pattern started deforming from 500 μm to 550 μm. More volume of air was added into channel, the width was raised to 800 μm. Moreover, the depth of pattern was declined from 80 μm to 70 μm. FIG. 3 shows the mechanism of soft robotics micromold. One purpose for the deformation of pattern is decreasing the depth in order to push microparticles out. The other purpose, also the main purpose is that PBS or medium can flow into wells in order to float microparticles out (FIG. 2c).

Comparison between Soft Robotics Micromold and Other Existing Methods. Soft and fragile are the main properties of collagen. Therefore, collagen would be broken easily when these microparticles were pushed out strongly. A major advantage of this soft mechanic device over other existing methods for generating collagen microparticles is that the shape and size were maintained the same as the pattern on the mold. The size of microparticles can be small as 100 to 300 μm. FIG. 2c shows that collagen microparticles were released from mold via soft mechanic device. The shape was the same as the pattern on the mold without breaking. Moreover, PBS would diffuse inside and even flow in the space, which was produced by air injection. On the other hand, collagen microparticles would not be stuck on the surface of PDMS mold after diffusion of PBS. FIG. 2d shows the morphology of collagen microparticles, which is launched by hand. Although collagen microparticles can be released from mold, the shape of particle was broken. The main reason is that collagen would be stuck on the surface of PDMS mold. If the force for bending was too strong, some parts of collagen particles would be remained on the PDMS surface. Finally, the damage would be shown on the collagen microparticles. Evidently, soft mechanic device can control the force for deformation in order to prevent the break and maintain the integrity of microparticles.

Also, three different materials were used, alginate and PEGDA, in order to synthesize the microparticles with different shapes. These three particles can be synthesized in soft robotics mold. And the size and shape of microparticles are similar with the mold. FIGS. 4a to 4i show various fabricated 2D structures (square, cross) and 3D structures (roller slide). Microparticles with 2D structure made from SRM can be created. In soft robotics micromold, the most important thing is that 3D microparticles can also be produced. The micromold and PEGDA microparticles with roller-slide shape are shown in FIGS. 4g and 4h. The scale of Roller-slide-like microparticles is about 500 um in the middle and 100 um on the top and bottom part. Conventional methods are hard to build microparticle with 3D structure. Normally, the thickness of microparticles is determined by the exposure time for photopolymerization or the diffusion time for chemo-polymerization. However, the height of microparticles is normally straight. If z-direction of microparticles is not straight, patterns would be patterned and aligned layer by layer. In soft robotics micromold, alginate (chemo-polymerization) or PEGDA (photoplymerization) can also construct 3D structures. The size and the shape are similar to the mold without any damage.

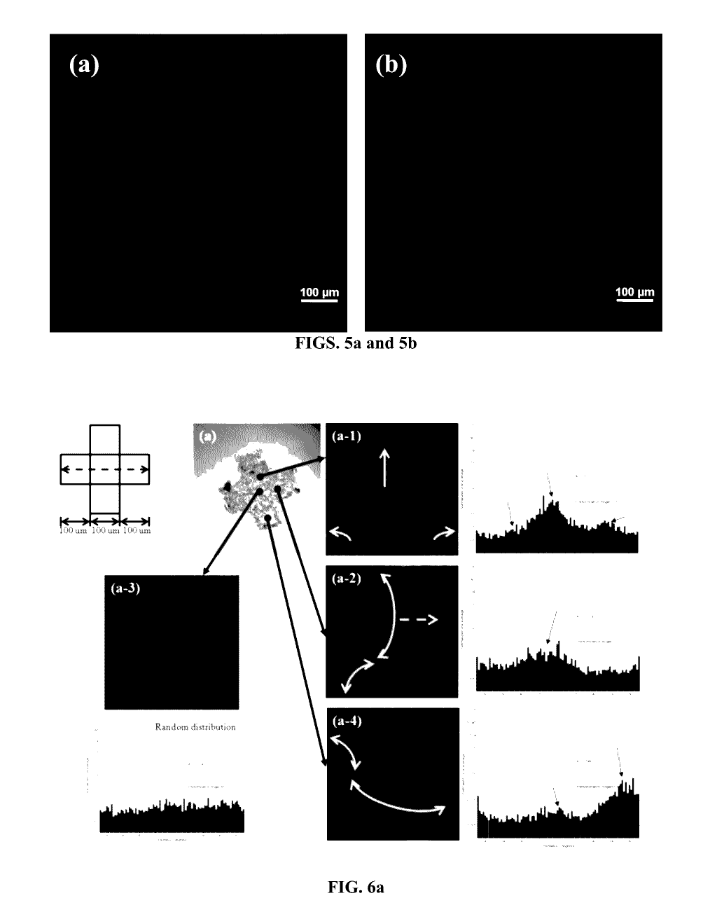

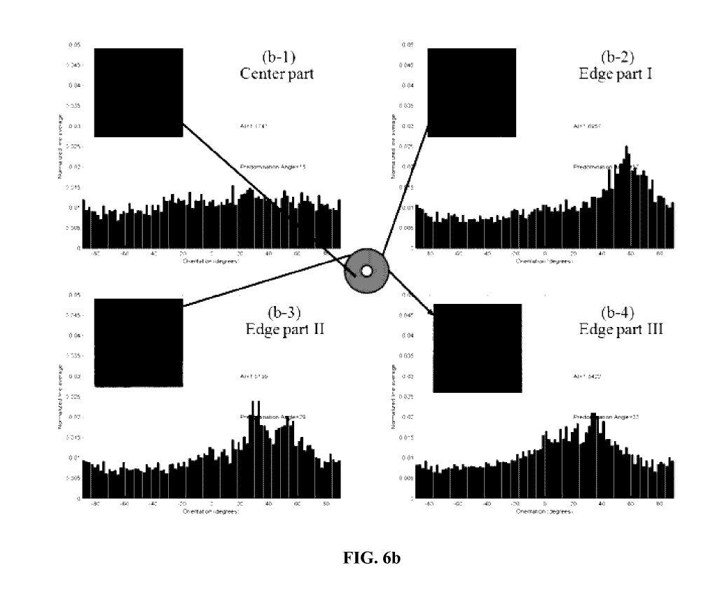

Alignment in Collagen Microparticles. FIGS. 4a to 4i present the morphology of collagen microparticles with square shape (FIGS. 4a, 4b), cross shape (FIGS. 4b, 4c) and bullet shape (FIG. 4f). The size and the shape are similar to the mold (FIGS. 4d, 4e) without any defect. Moreover, collagen microparticles with high aspect ratio (Height: 600 um; Width: 100 um) can also be fabricated by SRM (FIGS. 5a and 5b). The most important thing for collagen microparticles is that the alignment is shown in collagen microparticles, especially at the region of edge. Matrix fiber orientation distribution was analyzed by a MATLAB® routine based on a fast Fourier transform algorithm. Possible values of AI ranged from 4.55 for strong alignment (i.e., parallel fibers) to 1.00 for random alignment. FIGS. 6Aa to 6Aa-4 and 6Bb-1 to 6Bb-4 show second harmonic generation images. At edge area (FIG. 6Aa-1), collagen fiber would be aligned toward three different directions, 0° (middle), 45° (right), and −45° (left), respectively. This result is also confirmed by the calculation of MATLAB. The major peaks were presented at around 0°, 45°, and −45°, which matched the result showing on SHG image. Alignment Index (AI) in this region is 1.5713, which means the collagen fibril alignment at the edge area. Collagen fibril alignments are also shown at other two edge regions, and the major peaks are demonstrated around 0° to −40° and AIs are 1.3514 and 1.5491, respectively. However, the center part would not indicate any collagen orientation obvious, no matter what analytic tools are used, because the force cannot transfer to the middle section. On the other hand, when the micromold starts deforming, the edge part would be affected first. As the force of deformation increases, the deformation at the center part would start expanding. Thus, the collagen fibril alignment is random angle distribution. The same situation is shown at the donut shape. At the edge I, II, and III, collagen fibril alignments are exhibited at these three regions. Also, alignment index indicated that collagen fiber at three regions is aligned because AIs are 1.6957, 1.5199, and 1.5422, respectively. The angle of fiber orientation is about 25˜57°. These results indicate that collagen microparticles making from SRM can be induced fibril alignment at the edge area.

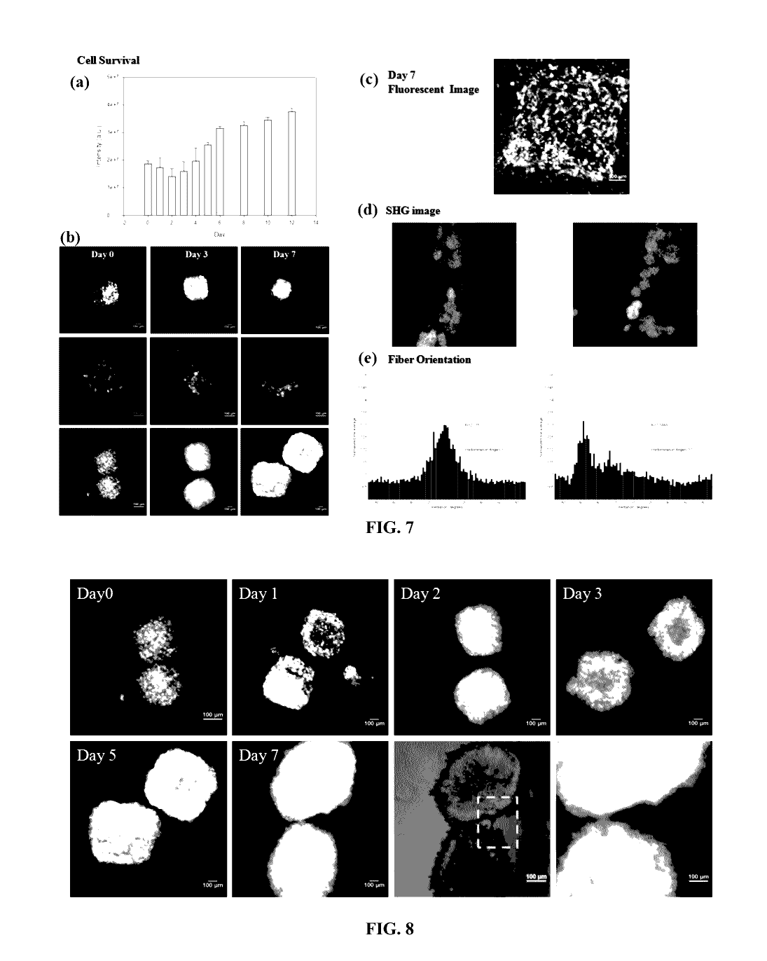

Cell-Based Microparticles/Encapsulated Cell Viability. FIGS. 7a to 7e show that the MDA-MB-231 cell line encapsulated in collagen microparticles. It was found that the MDA-MB-231 cells survive in collagen microparticles in excess of 14 days. When encapsulated microparticles incubated until 7 days, the lonely microparticles with cells will start shrinking. Once two encapsulated microparticles approached, microparticles would expand first and then start deforming. After 7 days, the shape of microparticles would not maintain the original shape and continue expanding in order to express intertumoral action (FIG. 8). On the other hand, these two microparticles can be considered as two tissues in the body, cancer cells would grow in the beginning. Therefore, cancer cells would stay and capture the fiber in order to stabilize the surroundings. As the number of cancer cells is enough and stabilized already, cancer cells would start migrating to other tissues (other microparticles). Therefore, ECM would start expanding in order to connect with other tissues. FIGS. 7c, 7d and 7e show the results of encapsulated collagen microparticles, when cancer cells existed inside the collagen, MDA-MB-231 started remodeling collagen fiber in order to migrate. From SHG image and its analytic data, collagen fibril alignment can be observed obviously. Moreover, AI would increase from 1.3491 (low degree of aligned orientation) to 2.035 (high degree of aligned orientation). Thus, these results indicate that we can use SRM to produce different shapes and sizes of collagen particles in order to mimic 3D collagen structure for discovering the interaction or signal transferring between different artificial tissues.

The present inventors demonstrate herein the fabrication of soft robotics micromold (SRM) system in order to produce collagen microparticles with different shapes and sizes. The SRM was used to produce cross, square-like (2D structure) and bullet, roller-slide (3D structure) hydrogel microparticles. The size of particles is from 100 μm to 1,000 μm, and the thickness can be determined the height of the mold (75 μm (low aspect ratio) to 600 μm (high aspect ratio)), which is made by lithography process.

Using soft robotics micromold system, we could produce collagen microparticles. The shape and the size of microparticles is exactly the same as the shape on the mold. Also, collagen microparticles were not damaged by the soft robotics micromold when released from the mold.

MDA-231 cells were mixed with collagen solution and cell-encapsulated microparticles were synthesized via SRM system. For over 14 days, MDA-231 survived well in the collagen particles and cell-encapsulated microparticles. While two microparticles were put together, the intertumoral interaction would be observed between these two cell-encapsulated microparticles. Second harmonic generation microscopy was used to detect the collagen and cancer cell. A breast cancer cell was shown to attach along the fibers and remodel along the orientation of fiber in order to migrate. The present invention can be used to make specific shapes of microparticles for use in tissue engineering.

### Example 2

### SRM Platform to Produce Islet Cell Encapsulated Collagen Microparticles

The present invention can be used to develop a molding platform for the high throughput production of collagen microparticles encapsulating beta cells and investigating its application for diabetes treatment in mice.

A SRM is a micro scale surface template structures to mold liquid collagen and millimeter scale pneumatic channel actuators to extract gelled collagen microparticles. These microparticles mimic native islet and can be implanted without inducing an inflammatory response. Important factor to consider include: [1] wetting morphologies of surface structures and [2] pneumatic channel actuator dynamics. Rapid solution dispensing to micro-scale templates is preferred to improve microparticle production throughput. The deformation mechanism of platforms can be optimized to improve the collagen microparticle extraction process that allows efficiently generating large numbers of microparticles for in-vivo mice studies. Although there are few alternatives to produce collagen microparticles, such as bottomless mold and microfluidic flow focusing, no existing methods are able to produce a large number of collagen microparticles reproducibly and rapidly without damaging them.

The SRM platform enables high throughput production of collagen microparticles to house functional beta cells as a source of insulin, e.g., in diabetic mice in vivo. The SRM diabetes platform can also be used elsewhere in the medical field, such as wrinkle filler, osteoarthritis of the knee, arthritis treatment, and rapid wound tissue heal. Applications of the SRM can be used to making three dimensional bio-microstructures from different materials and to target different tissues or medical conditions.

### Example 3

### SRM to Produce Islet Cell Encapsulated Collagen Microparticles for Treatment of Diabetes

Expanding on Example 2, the present inventors next produced SRM containing functional beta islet cells. Insulin produced by pancreatic beta cells is a vital hormone that drives blood glucose into cells and keep blood glucose normal. Insulin deficiency allows blood glucose to go up, leading to diabetes. Type 1 diabetes (T1D) is the result of absolute insulin deficiency and type 2 diabetes (T2D) is caused by relative insulin deficiency in the presence of increased demand for insulin in an insulin-resistant state that occurs, e.g., in obesity. Insulin therapy is the standard treatment for T1D, and an important mode of treatment for some T2D individuals. Human islet transplantation has been used to reverse T1D. The major limitations of islet transplantation is the shortage of human islets, and the requirement for chronic immunosuppressive therapy, which is toxic not only to the transplant recipient, but also the transplanted islets. About 35 years ago, Lim and Sun, Science 210: 908-910, 1980, first reported the use of microencapsulated islets, an approach that would solve these limitations by protecting the transplanted islets from the immune cells (making immunosuppression unnecessary), as well as allowing the use of nonhuman islets which would alleviate the shortage of human islets. Despite progress over the last decades, the lack of suitable microparticles to protect and support the foreign islets has continued to plague the field.

In this project, large number of collagen microparticles were produced with the SRM platform to address the limitations encountered in previous studies. The SRM platform produces at least 30,000 collagen microparticles from a single batch. The high throughput production of collagen microparticles enables the characterization of their performance for animal studies, as linked to the long-term insulin secretion performance for the future of diabetes clinical trials.

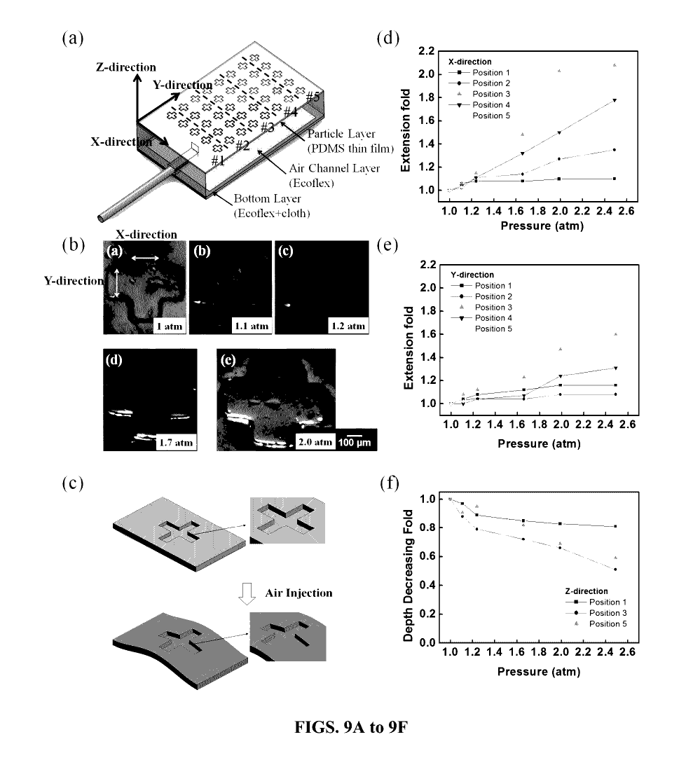

The first generation SRM platform can include three layers as shown in FIG. 9a, (1) micro-scale surface template layer for molding collagen, (2) pneumatic actuation layer to deform micro-templates to extract fragile collagen microparticles without damage, and (3) stress layer with cloth. Due to the high tensile stress of the cloth in the bottom layer, the platform deforms asymmetrically with the surface layer expanding the most while the bottom remains intact, which promotes the extraction of collagen microstructures from the surface template layer. FIG. 9b shows the microscopic images of cross-shaped micro-patterns on SRM platform as a function of the pressure application from 1 atm (no pressure) to 2.5 atm. FIG. 9c shows a schematic diagram of 3D deformation of micro-pattern. The scaled expansion rate defined as (w₀-w₁)/w₀, (w₀: the initial mold pattern width, w1: the mold pattern width after actuation). The expansion rates for cross shaped micro patterns in X, Y and Z directions at the position 1 to position 5 defined in FIG. 9a can be determined. The graph of the expansion rate of cross-shaped micro-pattern in X directional width as function of applied pressure is shown in FIG. 9d. The highest expansion rate of micro-pattern is observed at the position 3 at FIG. 9d. The expansion rate in Y direction at the position 1 to position 5 is shown in FIG. 9e. The averaged expansion rate from positions 1-5 in X directional width is 62.4% that is higher than one in Y directional width (60%). Because of this expansion, liquid can get into this space between collagen and template, which also improves the collagen extraction process. In addition to X, Y directional width, the depth of micro-pattern is reduced linearly from 100 μm to 56 μm as the function of applied pressure as shown in FIG. 9f. This Z directional extrusion from the bottom of micro-pattern increases the success rate of microparticle extraction process. The expansion rate of micro-patterns on the surface template layer is linearly proportional to the pressure application. Expansion and extrusion of micro-pattern improve the extraction of collagen microstructures from micro-patterns.

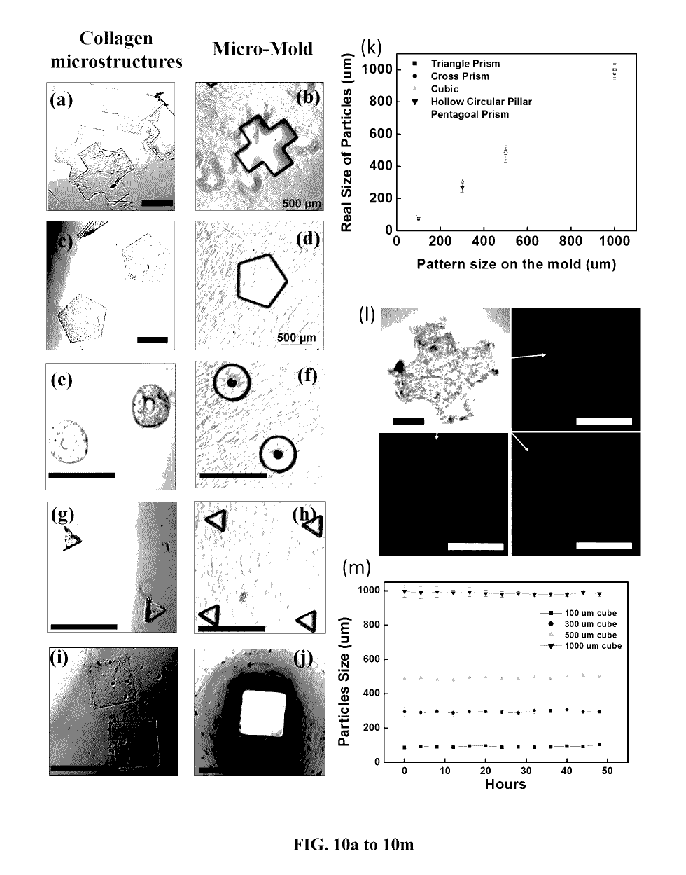

Collagen microparticles (20-30 particles from the single platform) were produced using the present invention. As shown in FIGS. 10a to 10o; microscopic images of collagen microstructures of cross column (FIG. 10a), pentagonal prism (FIG. 10c), hollow cylinder (FIG. 10e), triangular prism (FIG. 10g), and square pad (FIG. 10i) were produced via the SRM platform. In order to characterize the size reproducibility, the dimensions of the structures are compared to those of mold patterns (FIGS. 10b, 10d, 10f, 10h and 10j). The results show an approximately 96% accuracy for each microstructures ranging from 100 to 1000 μm, as shown in FIG. 10k. The reproducibility of collagen microstructures smaller than 100 μm is limited, as the collagen solution is not completely dispensed into micro patterns due to the importance of surface wettability in this length scale. The thickness of microstructures is set to 100 μm for all structures and is duplicated with more than 92% accuracy. FIG. 10l shows the result of second harmonic generation (SHG) microscopic images of collagen micro-cross column to characterize their collagen fiber structures. The density of collagen fiber distribution is determined using Image J to be 86.57%, which is higher than that of collagen microspheres produced in microfluidic devices (62.45%). This matches the density range of collagen fiber in dermis, bone, tendon and ligament (72-94%). In addition, collagen microstructures produced by SRM platform maintain their dimensions and shapes, with no observable shrinkage or expansion in size over 50 hours (FIG. 10m). These collagen microstructures are stored in the 4° C. refrigerator for more than 60 days without any shape changes.

Compared to hand pushing gelled collagen particles from the micro-templates, the extraction forces in SRM platform are easily controlled by applying an incrementally larger volume of air into the pneumatic channel. The successful extraction rates of micro cross-collagen by the SRM platform and manual pushing approach are 95% and 15%, respectively, as shown in FIG. 10n. FIG. 10n further demonstrates the structural damages observed among those collagen microparticles extracted manually.

FIG. 10o presents the insulin concentrations secreted from beta cells encapsulated in collagen microparticles and cultured on surfaces (2D cell culture). These cells are exposed to different concentrations of glucose (2 mM, 15 mM, and 30 mM). The concentrations of secreted insulin are normalized by total DNA concentrations extracted from cells. Thus, the beta cells encapsulated in collagen microparticles can secrete insulin 4-6 times higher than those on cultured on petro-dishes. Collagen microparticles provide real tissue like 3D microenvironment that improves the cell functions.

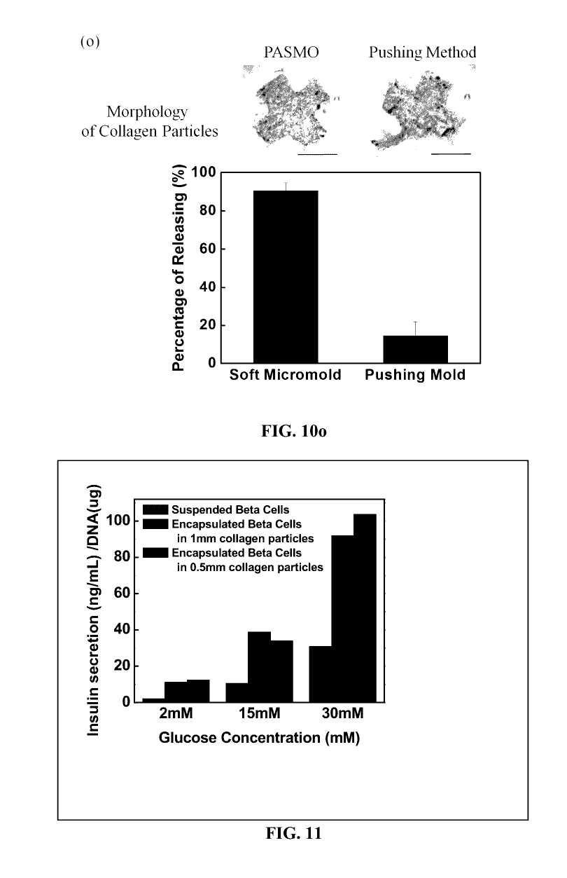

In addition, the relatively dense collagen microstructures can remain intact when they are injected into an agarose gel without being damaged, unlike their less dense collagen microsphere counterparts, as shown in FIG. 11. FIG. 11 demonstrates that collagen microstructures made by SRM platform are suitable for implant applications.

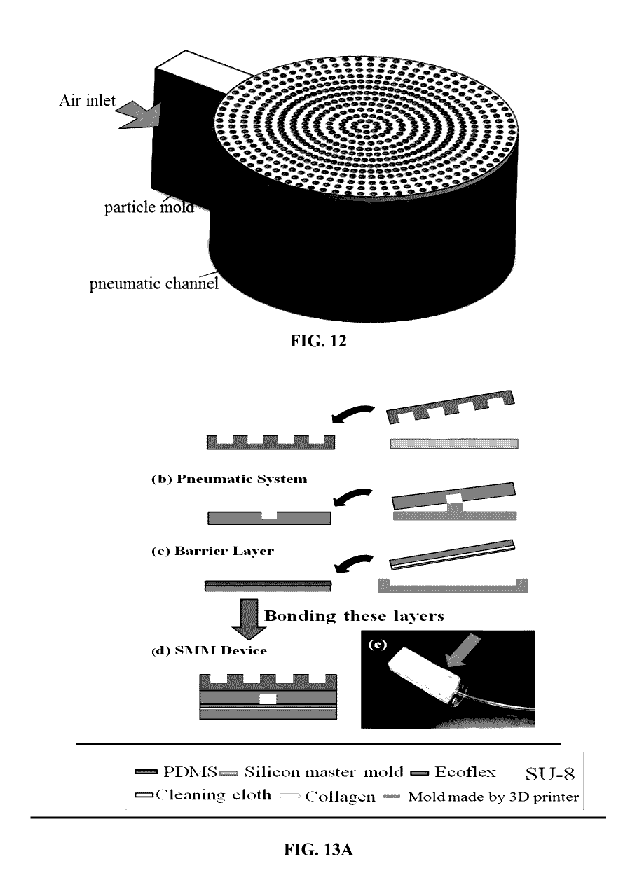

FIG. 12 shows a 3D view of the proposed SRM platform that contains two parts: (1) 30,000 micro scale surface template structures such as cross, triangle, oval, square circle and doughnuts shapes, and (2) millimeter scale fluidic channel networks for pneumatic actuations that deform and expand individual templates to extract collagen microparticles without damages. Air pressure is applied into this channel network from a syringe pump. To realize this platform, template wetting morphologies and pneumatic particle extraction actuators are investigated and integrated into the platform. The syringe pump to apply the pressure to the pneumatic actuator can control the sequence of collagen extraction process. Because this platform is mostly made of silicone, it is very economical and can be easily commercialized for many different applications, such as tissue engineering, dermal fillers or cartilage damage.

As shown in FIGS. 10a-10o, collagen microparticles are extracted directly into the cell culture media for in vitro studies. Beta cells are attached to nano scale fibers inside collagen microparticles. Collagen nanofiber structure provides (1) 3D tissue like environment for islet cells and (2) protective structure for beta cells from immune systems. Beta cells in real 3D tissue structure are normally bound to specific molecules in collagen nanofibers. This attachment induces specific biochemical signal transduction that is expected to improve the performance of insulin generation and glucose response. Because collagen is a natural biomaterial found in real tissue environment, it does not cause immune reaction that is caused by T cell and microphages. Because of these reasons, collagen microparticles encapsulating beta cells can provide a treatment option for diabetes. The collagen microparticles can be injected into mice, and their insulin secretion performance characterized.

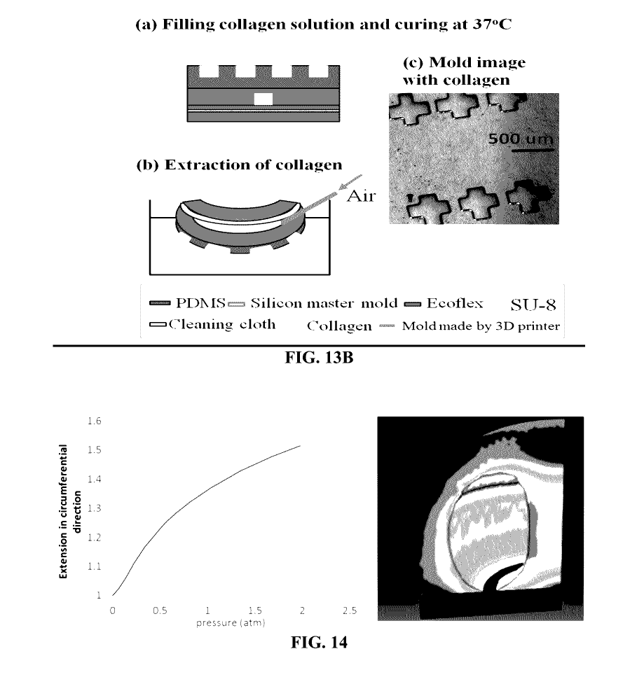

SRM platform fabrication process: The fabrication process of SRM platform is shown in FIGS. 13A-a to 13A-d. The master mold for the surface template layer is made via the standard photolithographic process with SU8 photoresist. A PDMS solution is spin-coated on this master mold and cured at 65° C. for 2 hours, then, peeled from the master-mold (FIG. 13A-a). The master molds for the pneumatic actuator and barrier layers are 3D-printed using an Envision tech 3D printer. Un-cured Ecoflex-30 is poured over a pneumatic actuator mold and cured for 4 hours at room temperature, and peeled from the mold (FIG. 13A-b). The width and depth of channel inside the pneumatic system layer are millimeter scale. A cleaning cloth with high tensile stress is placed inside the barrier layer mold; then, uncured Ecoflex-30 is dispensed over the cloth in the mold, cured at room temperature for four hours, and peeled from the mold (FIG. 13A-c). Surface template, pneumatic system and barrier layers are subsequently bonded at 65° C. for two hours with uncured Ecoflex-30 used as glue between layers (FIG. 13A-d).

Collagen microparticle production by SRM platform: The concentration of the human collagen type I, II, III and IV solution is set to 3.5-4.5 mg/mL that is reported as the condition conducive to making gelled collagen that mimics human tissue. The mixture rates of these collagen types are studied for collagen fiber density as well as insulin secretion performances of beta cells. The collagen type mixture solution is dispensed into the surface templates and gelled under the neutral condition (pH=7.4) at 37° C. for one hour (FIG. 13B-a). After the confirmation of gelled collagen, air pressure is applied to the pneumatic actuator from a syringe pump, which leads to the deformation of the SRM platform and the subsequent PBS liquid diffusion into small gaps between collagen and silicone wall. As a result, collagen microparticles are harvested in a PBS solution, as shown in FIG. 13B-b. The liquid diffusion process significantly improves the collagen microparticle extraction process. FIG. 13B-c) shows the mold image with collagen.

Analysis of surface wettability for dispensing collagen solution: As described hereinabove, the current collagen template design has been successfully implemented on a small scale, capable of producing fairly uniform microstructures. The current platform consists of approximately 30 templates per platform with a hydrophobic removable film that rids of excess liquid collagen prior to collagen solidification. Aqueous collagen is injected into templates by manually pipetting, which limits the scalability of this particular template design. In order to successfully scale up the production of collagen microstructures by orders of magnitudes, the free surface dynamics of aqueous collagen needs to be considered in two distinct ways: (1) spreading of liquid collagen into template cavities, and (2) formation of individual collage structures. Pouring aqueous collagen over the template with a hydrophobic PDMS film results in no collagen entry inside cavities, as the hydrophobicity of the film prevents the liquid collagen from spreading on the template and entering into cavities. Instead, collagen will most likely form individual droplets that pin on cavity corners (not shown); these effects are more pronounced at small length scales in which surface tension effects dominate. Pinning of the liquid-air contact line has been extensively studied and is known to depend strongly on the presence of local surface defects or sharp corners. Replacing the hydrophobic film with a hydrophilic one prevents aqueous collagen from forming droplets and allows the collagen to spread into hydrophilic cavities. This eliminates the need for pipetting collagen into individual cavities and, thereby, removes the clearest obstacle to scaling up the collagen production. Formation of uniform collagen microstructures depends on the clean removal of excess collagen outside the cavity as well as the control of the shape of the collagen interface inside the cavity. Removing the PDMS film upon liquid deposition ensures that most collagen liquid that has not entered the cavity be cleaned from the template. On the other hand, the shape of the collagen that remains inside the cavity strongly depends on the surface property of the cavity and the aspect ratio of the cavity cross-section. The surface property is quantified by the contact angle that collagen makes with a given surface; the smaller the contact angle is, the more “hydrophilic” the surface is. The contact angle can be then adjusted by treating the cavity surfaces with plasma or coating them with commercially available surfactants. For the given liquid volume inside the cavity, the cavity geometries may also yield various interfacial shapes of the collagen, ranging from “extended liquid filaments” to “overspilled droplets”. Based on the aforementioned physical principles, we will develop a mathematical model for the collagen shape inside the cavity by combining analytical solutions in simple cases (i.e., perfectly wetting or non-wetting liquid in a square cavity) and Surface Evolver simulations for more general geometries and a range of contact angle values. The quantitative model will enable a more informed template design for given applications.

FIGS. 13A-a to 13A-d shows a schematic diagram of fabrication process for Soft micro mold device. A. Fabricating process of (FIG. 13A-a) surface template layer (FIG. 13A-b) pneumatic system layer (FIG. 13A-c) barrier layer. (FIG. 13A-d) The bonding process of these three layers. (FIG. 13A-e) Photographic image of SMM device with TYGON® tubing.

FIGS. 13B-a to 13B-c show the Particles Releasing Process (FIG. 13B-a) Collagen microstructure fabrication process. Filling collagen solution into surface templates and curing at 37° C. (FIG. 13B-b) Collagen microstructure extraction process with pneumatic actuation. This is a side view as shown the arrow in FIGS. 13B-a to 13B-c show a microscopic image of micro scale surface templates of cross prism shape.

Numerical analyses for SRM platform responses and collagen microparticle extraction process: In order to understand and simulate responses of SRM platform under air pressure and extraction of collagen microparticles, constitutive models for various soft polymers that are considered in the SRM (see FIG. 12) and collagen particles. These constitutive models will be implemented within finite element (FE), which will help in the design of SRM, and simulating particle extraction process. Soft polymers considered in this project and collagen belong to a group of stimuli responsive soft polymers (PDMS, PEGDA, hydrogel, etc.) that can undergo large deformations (more than 500% straining) when subjected to various external stimuli, such as force, fluid/solvent, pH, thermal. During the particle extraction process the system is immersed in fluid/solvent and air pressure is injected through capillary channels; a coupled diffusion-deformation model is considered for modeling material responses of soft polymers and collagen. There have been limited models that incorporate large deformations and diffusion process in soft polymers, considering mainly incompressible material behaviors. Preliminary testing on the mechanical properties of Ecoflex-30, which is used for the mold, indicates that Ecoflex-30 is a compressible material. In this study, we consider a general diffusion-deformation model for polymer-solvent system by specifying a constitutive relation for the Gibbs potential that is consistent with thermodynamic requirements for compressible materials. The following Gibbs potential will be considered:

$${ρ}_{R} φ ( m , S ) = δ ( m ) + γ ( m ) tr ( S ) + \frac{β ( m )}{2} {( tr ( S ) )}^{2} + \frac{α ( m )}{2} tr ( {S}^{2} ) + HOT \qquad ( 1 )$$

where ρ_{R}, m, S are the density of solid body, density of fluid, and second Piola Kirchhoff stress tensor in the solid, respectively, δ, γ, β and α are the material constants that depend on the fluid concentration. The strain and chemical potential are determined from Eq. (1) as

$$E = {ρ}_{R} \frac{\partial φ}{\partial S}$$

$$and$$

$$μ = - {ρ}_{R} \frac{\partial φ}{\partial m} ,$$

respectively. For the diffusion model, we will consider the following form:

$$\frac{\partial m}{\partial t} = {C}_{1} Div ( A ( m ) Grad ( m ) ) + {C}_{2} Div ( Grad ( μ ( m , S ) ) ) \qquad ( 2 )$$

where C₁, C₂ and A are material constants. The above representations are general and can be adjusted for different soft polymers and collagen considered in this project by modifying the form of the Gibbs potential in Eq. (1). Initial forms in Eqs. (1) and (2) are taken based on current knowledge on the overall responses of soft materials, and experimental tests on select soft polymers and collagen can be used to improve the models.

The above constitutive models will be implemented in FE. The variational formulation for the diffusion equation and the equilibrium equation will be derived separately and then solved using a staggered solution procedure. Backward Euler will be used in discretizing the time domain for the diffusion equation. For the coupled problem, the equations will be solved iteratively. In this project FE simulations will be conducted prior to fabrication in order to examine the effect of different material and geometrical parameters (sizes and shapes of the molds that form collagen particles, see, e.g., FIGS. 10a-10j), and air pressure on the overall performance of collagen microparticles extraction. Different surface frictions between SRM platform and collagen particles can also be considered and different corners (sharp and curvy with several fillet radii) in the molds, and examine the extraction process in correlation with the design of SRM platform and immersion of fluid. This can provide additional insight into the important parameters in designing SRM and extraction of collagen microparticle. A preliminary design of SRM using Ecoflex-30 activated by air pressure is shown in FIG. 14A.

Physical property of 3D microenvironment for islet cells: The physical properties of collagen microparticles such as fiber density for beta cell encapsulation can be determined suing the present invention. Collagen elasticity is determined by collagen nanofiber density that is controlled by the collagen concentration and the mixture ratios of types (I, II, III, VI) of collagens. In addition, collagen gelled temperature and time are also important parameters to determine fiber density. The second harmonic imaging microscope can be used to investigate collagen fiber densities. Beta cells in collagen microparticles are placed on the 96-well plate and exposed to various concentrations of glucose. Their insulin secretions based on fiber densities are investigated. These insulin secretion results are compared to those obtained by 2D culture samples of islet cells without collagens as well as those encapsulated in PEGDA and Alginate.

Shape, dimension of collagen microparticles: The advantage of the proposed SRM platform is to produce various shapes of collagen microparticles, which cannot be achieved by any other currently existing particle production methods. The shape of microparticles is optimized for insulin secretion from beta cells. Cross, oval, triangle, pentagonal, square and bullet, doughnut shaped collagen microparticles encapsulating beta cells are produced by the SRM platform and the concentration of insulin secretion are characterized for 30 days.

Chemical and biological properties of 3D microenvironment for beta cells: The chemical and biological properties of collagen microparticles as 3D microenvironment for beta cells is characterized to make it as close as real tissue environment for insulin secretion. Islets and a collection of a combination of alpha cells and delta cells and other type of cells existing in pancreas, are the best choice to co-culture with any beta cells in collagen microparticles to enhance their performance. The volume of islet and beta cells in single collagen microparticles can be optimized.

Different types of cells can be co-cultured with beta cells in addition to islet such as fibroblast. These experiments are executed first in vitro. In addition to beta cells, micro scale poly(lactic-co-glycolic acid) (PLGA) or PEGDA can be used as molecular delivery carriers. PLGA and PEGDA microparticles in the diameter range between 4 μm to 200 μm have been fabricated by the microfluidic flow focusing devices. Various chemicals such as growth factors or cytokine are incorporated in microparticles (not shown) to stimulate beta cells. Chemical compounds contained in microparticles are optimized in this study and used for animal experiment. In addition, laminin can be included in collagen microparticles.

The conditions of the 3D microenvironment can be optimized for beta cells. The skilled artisan, following the present invention, can determine how to optimize keeping beta cells healthy and alive in vivo. PLGA microparticles, Poly(lactic acid) PLA and/or or Poly ethylene oxide (PEG), or direct molecules can also be included in collagen microparticles.

Testing of collagen microparticles in diabetic mice in vivo: C57BL/6 mouse pancreatic islet cells are isolated using standard procedures by the present inventors. Alternatively, Neurogenin3 can be used for in vivo transdetermination of hepatic progenitors cells into islet-like structures but not transdifferentiation of hepatocytes. Sustained expression of the transcription factor GLIS3 is required for normal beta cell function in adults, and embedding them in collagen microparticles during the gelling procedure will increase cell survival. Beta cell encapsulated microparticles have been readily implanted under the kidney capsule of normal and streptozotocin (STZ) induced diabetic mice. The efficacy of implanted encapsulated beta cells in reversing diabetes can be compared as determined by plasma blood glucose (morning random non-fasting, as well as after a 4-hour fast) with concomitant plasma insulin, complemented by glucose tolerance tests (GTT) again measuring both glucose and insulin. The present invention can be compared to the transplantation of collagen microparticle-encapsulated mouse islet with non-encapsulated mouse islet in reversing diabetes when implanted into STZ-induced diabetic mice, in terms of durability and efficacy of diabetes reversal. In another example, islet cells from Spargue Dawley rats can be isolated and the efficacy of the islets vs. C57BL/6 islets compared (free as well as micro-encapsulated) to normalize blood glucose in STZ-diabetic C57BL/6 recipients. Finally, the efficacy of C57BL/6 islets (free or microencapsulated) can be determined for reversing hyperglycemia in spontaneously diabetic NOD mice (which develop autoimmune diabetes). These latter studies can shed light on the efficacy of xenotransplantation of collagen microparticle-encapsulated islets in recipients of spontaneous autoimmune diabetes. The capacity of collagen microparticles to enable xenotransplanted islets to survive and reverse diabetes in autoimmune models will provide a reasonable predication whether a similar approach would allow human type 1 diabetic individuals to receive islet xenografts, e.g., from pigs.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Stevens, M. M.; George, J. H. Exploring and Engineering the Cell Surface Interface. Science 2005, 310, 1135-1138.
2. Sungmin Hong, Hui-Ju Hsu, Roland Kaunas, Jun Kameoka. Collagen microsphere production on a chip. Lab Chip. 2012 June; 12: 3277-3280.
3. Dhananjay Dendukuri, Daniel C. Pregibon, Jesse Collins, T. Alan Hatton and Patrick S. Doyle. Continuous-flow lithography for high throughput microparticle synthesis. Nature Material. 2006 April; 5: 365-369.
4. Dhananjay Dendukuri, Shelley S. Gu, Daniel C. Pregibon, T. Alan Hatton and Patrick S. Doyle. Stop-flow lithography in a microfluidic device. Lab Chip. 2007 May; 7: 818-828.
5. Alison P. McGuigan, Derek A. Bruzewicz, Ana Glavan, Manish Butte, George M. Whitesides. Cell encapsulation in sub-mm sized gel modules using replica molding. PLoS ONE. 2008 May; 3: e2258.
6. Stephanie E. A. Gratton, Patrick D. Pohlhaus, Jin Lee, Ji Guo, Moo J. Cho, and Joseph M. DeSimone. Nanofabricated particles for engineered drug therapies: A preliminary biodistribution study of PRINT™ nanoparticles. J. Control Release. 2007 June; 121: 10-18.

What is claimed is:

1. A method for producing an engineered tissue construct comprising:
providing a mold comprising one or more openings, the mold being at least partially elastic and the one or more openings having a pre-determined shape; and
extruding through the one or more openings in the mold a biocompatible gel-forming macromer to form a hydrogel using a mechanical force sufficient to extrude the hydrogel.

2. The method of claim 1, further comprising the step of isolating the extruded biocompatible hydrogel forms from the mold, wherein deformation of the mold causes the hydrogel in the pre-determined shape to be released from the mold.

3. The method of claim 1, wherein the mold comprises at least one of paper, cellulose, polydimethylsiloxane, rubber, plastic, polyethylene glycol, polytetrafluoroethylene (PTFE), polyphenyl ether polymers, modified polyphenyl ether polymers, poly(phenyl ether), or polyphenyl polyether.

4. The method of claim 1, further comprising the step of exerting a mechanical force on a reservoir that comprises the biocompatible gel-forming macromer, wherein the mechanical force is at least one of direct mechanical, pneumatic, or hydraulic (water or oil) force, wherein the mechanical force is defined further as at least one of a vertical or a horizontal mechanical force.

5. The method of claim 1, wherein the pre-determined shape includes at least one of shapes that can be interlocked into larger forms, have male and female interlocking portions, interlocking bricks, tongue and groove, dovetail joints, irregular, triangular, square, rectangular, pyramidal, rhomboidal, cross-shaped, bullet-shaped, cubic shaped, a tetrapod, a multipod, an arbitrary and partially curved, or the shape is not spherical.

6. The method of claim 1, wherein the biocompatible gel-forming macromer is dextran, heparin, heparin sulfate, chondroitin sulfate, hyaluronic acid, alginate, gelatin, collagen, albumin, ovalbumin, polyaminoacid, a single, oligomeric or polymeric residue of glycolic acid, lactic acid, caprolactone, butyrolactone, valerolactone, or carbonate, or is aligned into fibrils after formation into the hydrogel.

7. The method of claim 1, wherein the hydrogel further comprises one or more cells selected from at least one of pancreatic beta cells, pancreatic islets, chondrocytes, bone marrow, hepatocytes, pluripotent stem cells, totipotent stem cells, hematopoietic cells, mesenchymal stem cells, neural stem cells, cardiac stem cells, kerotinocytes, fibroblasts, ligament cells, endothelial cells, lung cells, epithelial cells, smooth muscle cells, cardiac muscle cells, skeletal muscle cells, nerve cells, kidney cells, bladder cells, urothelial cells, skin cells, neurons, Schwann cells, thyroid cells, reproductive cells, or bone-forming cells, or that are autologous to a subject for implantation, drug screening or tumor drug screening.

8. The method of claim 1, wherein the hydrogel further comprises one or more biologically active materials selected from at least one of a synthetic inorganic compound, an organic compound, a protein, a peptide, a polysaccharide, a lipid, a ganglioside, a nucleic acid, a growth factor, an antibody, a receptor, a lectin, a biological scaffold, a drug, a chemical, a chemotactic factor, one or more biologically active materials selected from at least one of a synthetic inorganic compound, an organic compound, a protein, a peptide, a polysaccharide, a lipid, a ganglioside, a nucleic acid, a growth factor, an antibody, a receptor, a lectin, a biological scaffold, a drug, a chemical, or a chemotactic factor covalently bound to the hydrogel.

9. The method of claim 1, further comprising the step of imaging a shape for insertion of the hydrogel, making a mold having the shape and size of the pre-determined shape, and extruding a hydrogel through the mold to form a hydrogel with the same shape and size of the pre-determined shape.

10. The method of claim 1, wherein the hydrogel is defined further as selected from at least one of:
as a first extruded hydrogel is incubated with a first cell type and a second extruded hydrogel is incubated with a second cell type and the first and second hydrogels are incubated together after isolation; or
the hydrogel comprises polymer fibrils, electrospun fibrils; or
the hydrogel comprises glycosaminoglycans added into the interstitial spaces between two or more extruded hydrogels; or the extruded hydrogel is isolated and polymer fibrils, electrospun fibrils, or glycosaminoglycans are added into the interstitial spaces between two or more extruded hydrogels and fluid is pumped between the two or more extruded hydrogels to mimic interstitial flow; or
the hydrogel is an extruded hydrogel is further subjected to compressive or tensile forces; or
the hydrogel is defined further as two or more extruded hydrogels that are incubated in a rotating bioreactor; or
the hydrogel is defined further as two or more extruded hydrogels that are formed into a tissue system, wherein the tissue system mimics in vivo biological system, specific cell behaviors, cell migration, viability, angiogenesis, apoptosis, proliferation, differentiation, gene expression, protein synthesis, protein secretion, tissue formation, cancer drug screening and cancer drug screening.

11. A method for making an hydrogel having a pre-determined shape comprising:
providing a mold comprising one or more openings, the mold being at least partially elastic and the one or more openings having the pre-determined shape;
extruding through the one or more openings in the mold a biocompatible gel-forming macromer to form a hydrogel using a mechanical force sufficient to extrude the biocompatible gel-forming macromer; and
isolating the extruded biocompatible hydrogel forms from the mold, wherein deformation of the mold causes the hydrogel in the predefined shape to be released from the mold.

12. The method of claim 11, wherein the mechanical force is defined further as at least one of a vertical or a horizontal mechanical force.

13. The method of claim 11, wherein the mold comprises at least one of paper, cellulose, polydimethylsiloxane, rubber, plastic, polyethylene glycol, polytetrafluoroethylene (PTFE), polyphenyl ether polymers, modified polyphenyl ether polymers, poly(phenyl ether), or polyphenyl polyether.

14. The method of claim 11, wherein the mechanical force is exerted on a reservoir that comprises the biocompatible gel-forming macromer, wherein the mechanical force is at least one of direct mechanical, pneumatic, or hydraulic (water or oil) force.

15. The method of claim 11, wherein the pre-determined shape includes at least one of shapes that can be interlocked into larger forms, have male and female interlocking portions, interlocking bricks, tongue and groove, dovetail joints, irregular, triangular, square, rectangular, pyramidal, rhomboidal, cross-shaped, bullet-shaped, cubic shaped, a tetrapod, a multipod, an arbitrary and partially curved, or the pre-determined shape is not spherical.

16. The method of claim 11, wherein the biocompatible gel-forming macromer is dextran, heparin, heparin sulfate, chondroitin sulfate, hyaluronic acid, alginate, gelatin, collagen, albumin, ovalbumin, polyaminoacid, a single, oligomeric or polymeric residue of glycolic acid, lactic acid, caprolactone, butyrolactone, valerolactone, or carbonate, or is aligned into fibrils after formation into the hydrogel.

17. The method of claim 11, wherein the hydrogel further comprises one or more cells selected from at least one of pancreatic beta cells, pancreatic islets, chondrocytes, bone marrow, hepatocytes, pluripotent stem cells, totipotent stem cells, hematopoietic cells, mesenchymal stem cells, neural stem cells, cardiac stem cells, kerotinocytes, fibroblasts, ligament cells, endothelial cells, lung cells, epithelial cells, smooth muscle cells, cardiac muscle cells, skeletal muscle cells, nerve cells, kidney cells, bladder cells, urothelial cells, skin cells, neurons, Schwann cells, thyroid cells, reproductive cells, or bone-forming cells, or that are autologous to a subject for implantation, drug screening or tumor drug screening.

18. The method of claim 11, wherein the hydrogel further comprises one or more biologically active materials selected from at least one of a synthetic inorganic compound, an organic compound, a protein, a peptide, a polysaccharide, a lipid, a ganglioside, a nucleic acid, a growth factor, an antibody, a receptor, a lectin, a biological scaffold, a drug, a chemical, a chemotactic factor, one or more biologically active materials selected from at least one of a synthetic inorganic compound, an organic compound, a protein, a peptide, a polysaccharide, a lipid, a ganglioside, a nucleic acid, a growth factor, an antibody, a receptor, a lectin, a biological scaffold, a drug, a chemical, or a chemotactic factor covalently bound to the hydrogel.

19. The method of claim 11, further comprising the step of imaging the pre-determined shape, making a mold having the shape and size of the pre-determined shape, and extruding a hydrogel through the mold to form a hydrogel with the same shape and size of the pre-determined shape.

20. A soft robotics micromold comprising:
a particle layer comprising one or more openings having a predetermined shape for making a hydrogel, wherein the particle layer maintains the predetermined shape but is also flexible, and optionally is a photopolymerizable polymer;
a fluid-channel layer disposed adjacent the particle layer, wherein the fluid-channel layer is constructed by a three-dimensional printer and comprises an elastic materials, the fluid channel layer comprising one or more channels that can be filled with a gas or a liquid that can displace a polymeric shape formed in the one or more openings; and
a bottom layer disposed adjacent the fluid-channel layer and opposite the particle layer, wherein the bottom layer is less elastic than the particle layer.

21. The micromold of claim 20, wherein the fluid-channel layer comprises at least one or a paper, cellulose, polydimethylsiloxane, rubber, plastic, polyethylene glycol, polytetrafluoroethylene (PTFE), polyphenyl ether polymers, modified polyphenyl ether polymers, poly(phenyl ether), or polyphenyl polyether.

22. The micromold of claim 20, wherein a hydrogel formed in the one or more openings comprises at least one of dextran, heparin, heparin sulfate, chondroitin sulfate, hyaluronic acid, alginate, gelatin, collagen, albumin, ovalbumin, polyaminoacid, a single, oligomeric or polymeric residue of glycolic acid, lactic acid, caprolactone, butyrolactone, valerolactone, or carbonate.

23. A method of making a soft robotics micromold comprising:
providing a particle layer comprising one or more openings having a predetermined shape for making a hydrogel, wherein the particle layer maintains the predetermined shape but is also flexible, wherein the particle layer is optionally a photopolymerizable polymer, and the hydrogel formed in the one or more openings comprises at least one of dextran, heparin, heparin sulfate, chondroitin sulfate, hyaluronic acid, alginate, gelatin, collagen, albumin, ovalbumin, polyaminoacid, a single, oligomeric or polymeric residue of glycolic acid, lactic acid, caprolactone, butyrolactone, valerolactone, or carbonate;
depositing a fluid-channel layer disposed adjacent the particle layer, wherein the fluid-channel layer is constructed by a three-dimensional printer and comprises an elastic materials, the fluid channel layer comprising one or more channels that can be filled with a gas or a liquid that can displace a polymeric shape formed in the one or more openings, wherein the fluid-channel layer comprises at least one or a paper, cellulose, polydimethylsiloxane, rubber, plastic, polyethylene glycol, polytetrafluoroethylene (PTFE), polyphenyl ether polymers, modified polyphenyl ether polymers, poly(phenyl ether), or polyphenyl polyether; and
attaching a bottom layer to the fluid-channel layer and opposite the particle layer, wherein the bottom layer is less elastic than the particle layer.
